(19) United States
(12) Patent Application Publication
(10) Pub. No.: US 2024/0325522 A1
(43) Pub. Date: Oct. 3, 2024

(54) RNA ADSORBED ONTO LIPID NANO-EMULSION PARTICLES AND ITS FORMULATIONS

(71) Applicant: Gennova Biopharmaceuticals Limited, Maharastra (IN)

(72) Inventors: Sanjay SINGH, Maharastra (IN); Swarnendu KAVIRAJ, Maharastra (IN); Ajay SINGH, Maharastra (IN); Arjun Singh RAGHUWANSHI, Maharastra (IN); Pavan KARDILE, Maharastra (IN); Shalu SHUKLA, Maharastra (IN); Aishwarya KULKARNI, Maharastra (IN); Praveen AGRA WAL, Maharastra (IN); Sunil RAUT, Maharastra (IN)

(21) Appl. No.: 18/578,881

(22) PCT Filed: Jul. 8, 2022
(86) PCT No.: PCT/IN2022/050624

§ 371 Date: Jan. 12, 2024

(30) Foreign Application Priority Data
Jul. 13, 2021 (IN) 202121031414

(51) Int. Cl. A61K 39/215; A61K 9/107; A61K 39/00

(52) U.S. Cl. CPC A61K 39/215; A61K 9/1075; A61K 2039/53; A61K 2039/55572; A61K 2039/6018

(57) ABSTRACT

RNA adsorbed onto lipid nano-emulsion particles and its formulations. The present invention relates, a method of preparing a liquid formulation of RNA complexed with lipid nano-emulsion particles or nano-carriers. It particularly provides a method for preparation of the RNA adsorbed onto lipid nano-emulsion particles in liquid and the formulations of said RNA complexes as such.

### THE FIELD OF INVENTION

The present invention relates, a method of preparing a liquid formulation of RNA complexed with lipid nano-emulsion particles or nano-carriers. It particularly provides a method for preparation of the RNA adsorbed onto lipid nano-emulsion particles in liquid and the formulations of said RNA complexes as such.

### BACKGROUND

Recently, in many therapeutically relevant pharmaceutical applications, nucleic acids, as such, are used for therapeutic and diagnostic purposes. As an example, in recent years in the field of RNA-based therapy promising results have been achieved. Herein various types of RNA molecules are regarded as important tools for gene therapy as well as prophylactic and therapeutic vaccination against many infectious and malignant diseases.

Nucleic acids, both DNA and RNA, have been used widely in gene therapy, either in naked or in complexed forms. The use of RNA is advantageous in modern molecular medicine, having some superior properties over the use of DNA. As is known, transfection of DNA molecules may lead to serious complications and these risks do not occur if particularly mRNAs are used instead of DNA. An advantage of using RNA rather than DNA is that no virus-derived promoter element has to be administered in vivo and no integration into the genome may occur and the RNA does not need to travel to the nucleus for the expression.

The use of RNAs, as such, as pharmaceutical agents has been limited due to its sensitivity to degradation and problems of the delivery across the cell membranes upon injection in the body of animals or human subjects. The RNA molecules are inherently unstable due to its structural properties and degrade fast in general conditions if not stabilized.

Thus, an object of the present invention is to provide a method for the effective delivery of disease modifying mRNA molecules in combination with novel lipid nano-emulsion particles [also called nano-carriers] forming a liquid pharmaceutical formulation, which is clinical effective, safe, scalable, and is also time- and cost-efficient. Therefore, an object of the invention is to provide a composition of said lipid nano-emulsion particles and a method of complexing mRNA molecules of interest with the particles, and such RNA molecules on being transported into a cell, a tissue or an organism generating the required therapeutic or immunological effects. A further object of the invention is to provide methods for the preparation of mRNA molecules and nano-carriers suitable for said formulations.

### DESCRIPTION

The present invention has the following aspects:
1. mRNA molecules and preparation thereof;
2. Lipid nano-emulsion particles or nano-carriers and preparation thereof; and
3. Preparation of a liquid formulations of said mRNA molecules adsorbed onto said nano-carriers.

In a first aspect, the present invention relates to the preparation of RNA or mRNA molecules capable of expressing of a protein or peptide when delivered inside a live cell using nano-carriers. The said RNA being of therapeutic or prophylactic nature is useful in the pharmaceutical applications. The said mRNAs having sequences of the genes of interest, which may relate to antigens derived from genes of viruses, bacteria or other microorganisms or higher organisms.

In a preferred embodiment, the said RNA or mRNA comprises from 50 to 50000 nucleotides, preferably having from 200 to 15000 nucleotides and more preferably having from 500 to 12000 nucleotides. Aside from messenger RNA, non-coding types such as ribosomal RNA or transfer RNA and other coding RNA molecules, such as viral RNA, retroviral RNA, self-replicating RNA, small interfering RNA, microRNA, small nuclear RNA, small-hairpin RNA or a combination thereof may be used in the invention disclosed herein. Further said coding or non-coding RNA may comprise modified RNA having enhanced properties like stability in vitro and in vivo. The said RNA modification may refer to chemical modifications comprising backbone modifications as well as sugar modifications or base modifications.

In the invention disclosed herein, the said RNA may be encoding a protein or a peptide or an antigen, which may be selected, without any restriction, from therapeutically active proteins or peptides, selected from adjuvant proteins, from tumour antigens, pathogenic antigens (e.g. selected, from animal antigens, from viral antigens, from protozoan antigens, from bacterial antigens), allergenic antigens, autoimmune antigens, or further antigens, from allergens, from antibodies, from immunostimulatory proteins or peptides or from any other protein or peptide suitable for a therapeutic application.

Herein, a modified RNA molecule may contain nucleotide analogues/modifications, e.g. backbone modifications, sugar modifications or base modifications. A modification in connection with the present invention is a modification, in which the capping of RNA molecules is done at the 5′-end using enzymes in vitro.

In a preferred embodiment, said liquid formulation of the invention comprises at least one RNA, wherein the RNA is an mRNA molecule, having at least one open reading frame, which encodes at least one peptide or protein. Further said modified RNA molecule is having two of more open reading frames for peptides or proteins, which aid in the replication of said RNA molecules in vivo [also called self-replicating mRNAs] and preferably, the sequence of the open reading frame in such an RNA molecule is modified as described herein.

In the present invention, the said RNA comprised in said composition comprises a 5′- and/or 3′ untranslated regions (5′-UTR or 3′-UTR, respectively). Preferably, the at least one RNA comprises at least one selected from the group consisting of a 5′-UTR, a 3′-UTR, a poly (A) sequence and/or a poly (C) sequence. More preferably, at least one RNA comprises a 5′-CAP structure.

In the present invention, a 5′-UTR is typically the part of an mRNA, which is located between the protein coding region and the 5′-terminus of the mRNA. A 5′-UTR of an mRNA is not translated into any amino acid sequence. The 5′-UTR sequence is generally encoded by the gene, which is transcribed into the respective mRNA during the gene expression process. In the context of the present invention, a 5′-UTR corresponds to the sequence of a mature mRNA, which is located 3′ to the promoter sequence and immediately 5′ to the start codon of the protein coding region.

In a further preferred embodiment, the said RNA of the invention comprises at least one 5′-UTR. More preferably, at least one RNA comprises a 5′-UTR, which comprises or consists of a nucleic acid sequence derived from the 5′-UTR of an Alpha virus gene. Preferably, at least one RNA comprises a 5′-UTR, which may be derivable from a gene that relates to an mRNA with an enhanced half-life. The nucleotide sequence of 5′-UTR element of an Alpha virus is, gene namely, ATAGGCGGCGCATGAGAGAAGCCCAGACCAATTACCTACCCAAA from the Venezuelan Equine Encephalitis Virus (VEEV) strain TC-83.

In the present invention, a 3′-UTR is typically the part of an mRNA, which is located between the protein coding region and the 3′-terminus of the mRNA. A 3′-UTR of an mRNA is not translated into any amino acid sequence. The 3′-UTR sequence is generally encoded by the gene, which is transcribed into the respective mRNA during the gene expression process. In the context of the present invention, a 3′-UTR corresponds to the sequence of a mature mRNA, which is located 3′ to the stop codon of the protein coding region, preferably immediately 3′ to the stop codon of the protein coding region, and which extends to the 5′-side of the 3′-terminus of the mRNA or of the poly (A) sequence, preferably to the nucleotide immediately 5′ to the poly (A) sequence.

In a further preferred embodiment, the said RNA of the invention comprises at least one 3′-UTR. More preferably, at least one RNA comprises a 3′-UTR, which comprises or consists of a nucleic acid sequence derived from the 3′-UTR of an Alpha virus gene. Preferably, at least one RNA comprises a 3′-UTR, which may be derivable from a gene that relates to an mRNA with an enhanced half-life. The nucleotide sequence of 3′-UTR element of an Alpha virus gene is, namely, GGTGTCAAAAACCGCGTGGACGTGGTTAACATCCCTGCTGGGAGGATCAGCCGTAA TTATTATAATTGGCTTGGTGCTGGCTACTATTGTGGCCATGTACGTGCTGACCAACC AGAAACATAATTGAATACAGCAGCAATTGGCAAGCTGCTTACATAGAACTCGCGGC GATTGGCATGCCGCCTTAAAATTTTTATTTTATTTTTTCTTTTCTTTTCCGAATCGGA TTTTGTTTTTAATATTTC from the Venezuelan Equine Encephalitis Virus (VEEV) strain TC-83.

Preferably, at least one 5′-UTR and at least one 3′-UTR act synergistically to increase protein production from the said RNA comprised in the liquid formulation of the invention, when delivered to the cells.

In a preferred embodiment, said RNA of the invention further comprises a poly (A) sequence. The length of the poly (A) sequence may vary. The poly (A) sequence may have a length of about 20 up to about 300 adenine nucleotides, preferably of about 40 to about 200 adenine nucleotides. Most preferably, the said RNA comprises a poly (A) sequence of about 40 to about 60 nucleotides, most preferably 45 adenine nucleotides.

In a preferred embodiment, for the synthesis of said RNA, a DNA template is prepared from a plasmid cultured in an E. coli cell line. The plasmid is isolated from the bacteria and enzymatically linearized to obtain the DNA template or alternatively said DNA template is obtained by the polymerase chain reaction using small amount of the plasmid or bacterial host harbouring said plasmid as the reaction source.

Once the template DNA is ready, in vitro transcription is performed in the presence of a suitable phage promoter, not limited to T7 RNA polymerase. The RNAse inhibitor may also be used to protect the RNA from degradation. Reaction also uses a pyrophosphtase enzyme that converts the insoluble pyrophosphate into inorganic phosphate, a by-product of in vitro transcription. The DNA template, enzyme mix and rNTPs are incubated under the appropriate conditions to yield the mRNA of a size between 500 and 50000 nucleotides.

After the synthesis of mRNA, the DNA template is degraded from the reaction mixture by DNAse enzymes in the presence of salts under the appropriate conditions. The next step is the mRNA protection by 5′ capping. This can be achieved by chemical conjugation or enzymatic reaction. Then the crude mRNA preparation is first purified by column chromatography in the flow-through mode, binding impurities to the resin while the mRNA flows through the resin. This flow through is collected for the next step of affinity chromatography to remove the similar impurities. The eluate from the affinity column is concentrated and diafiltered with the hollow fibre modules and further sterilized by membrane filters. This product is used for the complexing with nano-carriers.

In a second aspect, the present invention relates to lipid nano-emulsion particles (also called nano-carriers) and a method for preparing it in liquid. The said nano-carriers comprising properties of adsorbing single strand mRNA molecules on its surfaces and allowing delivery of said mRNA molecules across the cell membranes into the cells.

In a particularly preferred embodiment, the said nano-carriers comprises at least one cationic or polycationic lipid compound, preferably as defined herein, wherein the said cationic or polycationic compound are present in a complex with other components forming stable lipid nano-emulsion particles or nano-carriers. The said nano-carriers of the invention preferably comprises a cationic or polycationic lipid compound, preferably DOTAP (1,2-dioleoyl-3-trimethylammonium-propane), DDA (dimethyldioctadecylammonium) or similar cationic/polycationic lipids.

In a preferred embodiment, the term “nano-carrier(s)” typically refers to a composition of the lipid nano-emulsion particles [herein also cited as GNPs or its derivatives] comprising a cationic or polycationic compound and other components that supports the formation and stability of such complexes. The GNPs are also known as cationic nano-emulsions (CNEs) or cationic lipid nano-emulsions (CLNEs) in the art.

In a preferred embodiment, said nano-carriers have the average size, preferably in a range from 30 to 300 nm, more preferably from 50 to 200 nm. In a particularly preferred embodiment, the average size of the nano-carriers comprising or consisting of complexed RNA is from 50 to 100 nm.

In a preferred embodiment, said nano-carriers with or without RNA adsorbed onto it, have a poly dispersity index [PDI] relating to its size in a range from 0.150 to 0.300, more preferably from 0.170 to 0.230.

In a preferred embodiment, said nano-carriers comprising or consisting of a cationic or polycationic compound, have a zeta potential value in a range from −10 to −50 mV, more preferably from −25 to −35 mV.

In a preferred embodiment, said nano-carriers remain stable in a suitable solvent. Preferably, a solvent, which allows dissolution of said RNA and, further components, such as buffering agents, etc as defined herein. More preferably, the solvent is volatile with a boiling point of preferably below 120° C. In addition, the solvent is preferably non-toxic. Preferably, the solvent is an aqueous solution. In the case of an organic solvent, the solvent is preferably miscible with water. The solvent provided may comprise a buffer, preferably selected from a buffer as defined herein.

In a preferred embodiment, said nano-carriers provided may additionally contain at least one component selected, e.g., from immunostimulants, metal compounds or metal ions, surfactants, polymers or complexing agents, buffers, etc., or a combination thereof.

In a preferred embodiment, the said nano-carriers provided may additionally contain a further component selected from the group of surfactants. Such group may comprise, without being limited thereto, any surfactant suitable for the preparation of a pharmaceutical composition, preferably, without being limited thereto, polysorbate, sorbitan, etc.

In a preferred embodiment, said nano-carriers provided may additionally contain a further component selected from the group of non-specific immunostimulants. Such group may comprise, without being limited thereto, any non-specific immunostimulants suitable for the preparation of a pharmaceutical composition, preferably, without being limited thereto, squalene or any other similar compounds.

In a preferred embodiment, the said nano-carriers provided may additionally contain a further component selected from the group of specific immunostimulants. Such group may comprise, without being limited thereto, any specific immunostimulants suitable for the preparation of a pharmaceutical composition, preferably, without being limited thereto, monophosphoryl lipid-A [MPL] or glucopyranosyl lipid-A [GLA] or any other similar adjuvant compounds.

In a third aspect, the present invention relates a liquid formulation comprising an mRNA adsorbed onto lipid nano-emulsion particles or nano-carriers as described herein in below and said formulation comprising said mRNA at a concentration preferably between 0.1 and 1 mg/mL.

In a preferred embodiment, said RNA or mRNA comprised in the liquid formulation is complexed at least partially with a cationic or polycationic lipid contained in said nano-carriers. Partially means that only a part of the at least one RNA molecule is complexed with a cationic or polycationic compound and that the rest of the at least one RNA molecule is in non-complexed form (“free”). Preferably the ratio of complexed RNA to free RNA is between 25:1 (w/w) and 50:1 (w/w), more preferably is about 50:1 (w/w).

In a preferred embodiment, the relative integrity is preferably determined as the percentage of full-length RNA (i.e. non-degraded RNA) with respect to the total amount of RNA (i.e. full-length RNA and degraded RNA fragments (which appear as smears in gel electrophoresis images), preferably after deduction of background noise, for example, by using a software based densitometry. Preferably, the relative integrity of the said RNA in the liquid formulation of inventive method is at 80% and more preferable at least 90% after storage at freezing temperature for preferably at least six months.

In a preferred embodiment, the biological activity of the said RNA of the liquid formulation after storage at room temperature, preferably as defined above with respect to the relative integrity of the said RNA, is preferably at least 70%, more preferably at least 80% and most preferably at least 90% of the biological activity of the freshly prepared RNA. The biological activity is preferably determined by analysis of the amounts of protein expressed from reconstituted RNA and from freshly prepared RNA, respectively, e.g. after transfection into a mammalian cell line or into a subject. Alternatively, the biological activity may be determined by measuring the induction of an (adaptive or innate) immune response in a subject.

Further, the disclosed invention provides the use of the inventive method and products in the manufacture of a pharmaceutical preparation or a vaccine. According to an aspect of the present invention, a pharmaceutical formulation is provided, which comprises or consists of the liquid formulation obtainable by the inventive method. In a preferred embodiment, the inventive pharmaceutical formulation comprises at least one additional pharmaceutically acceptable ingredient, such as a pharmaceutically acceptable carrier and/or vehicle. The inventive pharmaceutical formulation may optionally be supplemented with further components as defined above with regard to the liquid formulation. The inventive pharmaceutical formulation is prepared as a whole by the inventive method.

Preferably, the inventive pharmaceutical formulation may be administered by parenteral injection, more preferably by subcutaneous, intravenous, intramuscular injection. Sterile injectable forms of the inventive pharmaceutical formulations may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent. These oil solutions or suspensions may also contain a long-chain alcohol diluent or dispersant, such as carboxymethylcellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable forms may also be used for the purposes of formulation of the invention.

The inventive pharmaceutical composition typically comprises a “safe and effective amount” of the components of the inventive pharmaceutical formulation as defined above, particularly of at least one RNA as comprised in the said formulation obtainable by the inventive method. As used herein, a “safe and effective amount” means an amount of the at least one RNA that is sufficient to significantly induce a positive modification of a disease or disorder as defined herein. At the same time, however, a “safe and effective amount” is small enough to avoid serious side-effects, that is to say to permit a sensible relationship between advantage and risk. The inventive pharmaceutical formulation may be used for human and also for veterinary medical purposes, preferably for human medical purposes, as a pharmaceutical formulation in general or as a vaccine.

According to a specific embodiment, the pharmaceutical formulation comprises an adjuvant. In this context, an adjuvant may be understood as any compound, which is suitable to initiate or increase an immune response of the innate immune system, i.e. a non-specific immune response. In other terms, when administered, the inventive vaccine preferably elicits an innate immune response due to the adjuvant, optionally contained therein. Preferably, such an adjuvant may be selected from an adjuvant known to a skilled person and suitable for the present case, i.e. supporting the induction of an innate immune response in a mammal. In this context, the adjuvant is preferably selected from compounds, which are known to be immune-stimulating due to their binding affinity (as ligands) to human toll-like receptor-4 [TLR4] like GLA [Glucopyranosyl Lipid Adjuvant] or MPL [Glucopyranosyl Lipid Adjuvant].

The present invention furthermore provides several applications and uses of the formulation obtainable by the disclosed method, the inventive pharmaceutical formulation, the inventive vaccine or the inventive kit or kit of parts.

### DESCRIPTION OF FIGURES

The figures shown in the following are merely illustrative and shall describe the present invention in a further way. These figures shall not be construed to limit the present invention thereto.

FIG. 1: Depicts the general structure of the pVEE plasmids [which is based on pMB1 plasmid] with self-replicating mRNA expressing cassettes for different antigens as disclosed in the invention disclosed.

FIG. 2: SEQ ID NO: 1, is the protein sequence corresponding to SARS-CoV-2 Spike Protein B.1 Variant with a D614G mutantion.

FIG. 2-1: SEQ ID NO: 2, is the protein sequence corresponding to SARS-CoV-2 Spike Protein Wuhan-Hu-1 variant [GISAID Accession No.: EPI_ISL_402125 Virus name: hCoV-19/Wuhan/Hu-1/2019 Collection date: 31 Dec. 2019].

FIG. 3: Depicts the quality of mRNA during IVT process. [A] shows different steps of IVT, IVT-A step: Lane 2 [2 h incubation], 3 [3 h] and 4 [4 h]; IVT-B step: Lane 5 and IVT-C step: Lane 6. [B] shows different steps of purification of mRNA, Lane 2 crude mRNA preparation from IVT-C, while Lane 10 is retentate finally obtained. [C] shows the final drug substance obtained from retentate, Lane 2. The molecular weight marker in all gels is the same having first bank of 9 kb, sixth band of 3 kb and 10th band of 0.5 kb of single strand RNA.

FIG. 3-1: Depicts the chromatographic profile of the purified mRNA batch sample as described in Example 3.

FIG. 4: Shows the SEC-HPLC profile of the purified SAR-CoV-2 spike protein mRNA construct of about 12 kb having retention time [RT] of about 8.15 minutes.

FIG. 5: Shows relative integrity of mRNA after adsorption onto lipid nano-emulsion particles or nano-carriers of different types. [A] shows the adsorption of mRNA molecules onto nano-carriers showing no movement of said mRNA molecules in electric field. [B] shows the RNAse protection of bound mRNA molecules to different nano-carriers. The bound mRNA molecules remain intact even after the RNAse treatment.

FIG. 5-1: Shows expression profile of naked mRNA and mRNA adsorbed or complexed onto the nano-carrier in the HEK 293T ells in vitro.

FIG. 6: Shows the immune response generated by vaccine formulations comprising the mRNA molecules and nano-carriers of the inventive method disclosed herein. The SARS-CoV-2 spike protein producing self-replicating mRNA constructs when adsorbed onto the nano-carriers, namely, GNP, GNP-M or GNP-G and injected into mice produced robust IgG responses.

FIG. 7: Shows the neutralising antibody response generated by vaccine formulations comprising the mRNA molecules and nano-carriers of the inventive method disclosed herein. The SARS-CoV-2 spike protein producing self-replicating mRNA constructs when adsorbed onto the nano-carriers, namely, GNP, GNP-M or GNP-G and injected into mice produced robust virus neutralising antibodies.

### EXAMPLES

The Examples shown below are illustrative, further describing the present invention and shall not be construed to limit it.

### Example 1: Plasmids for the Expression of mRNA Molecules

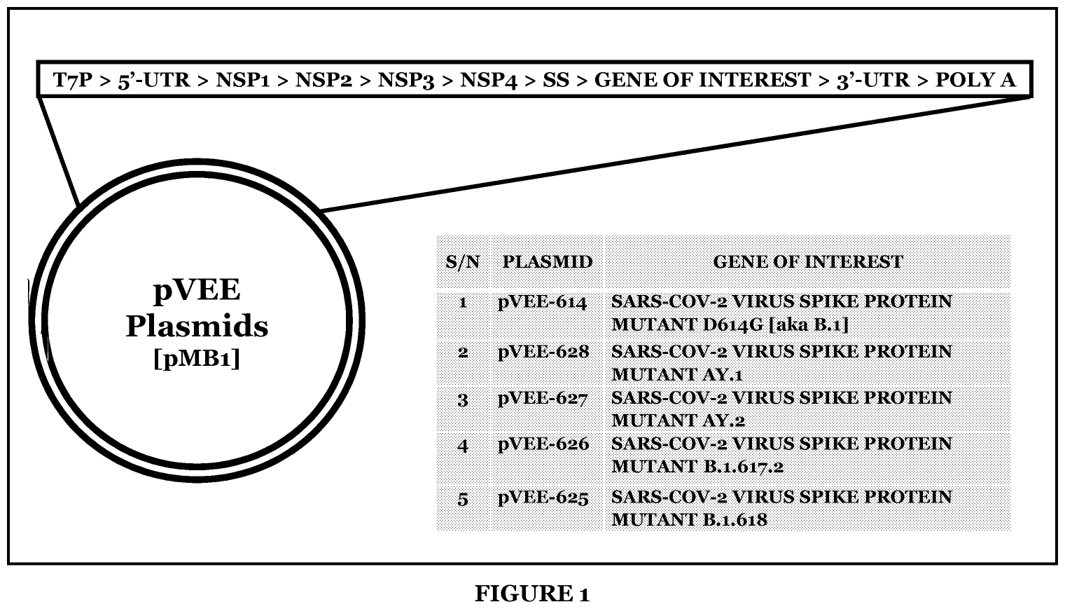

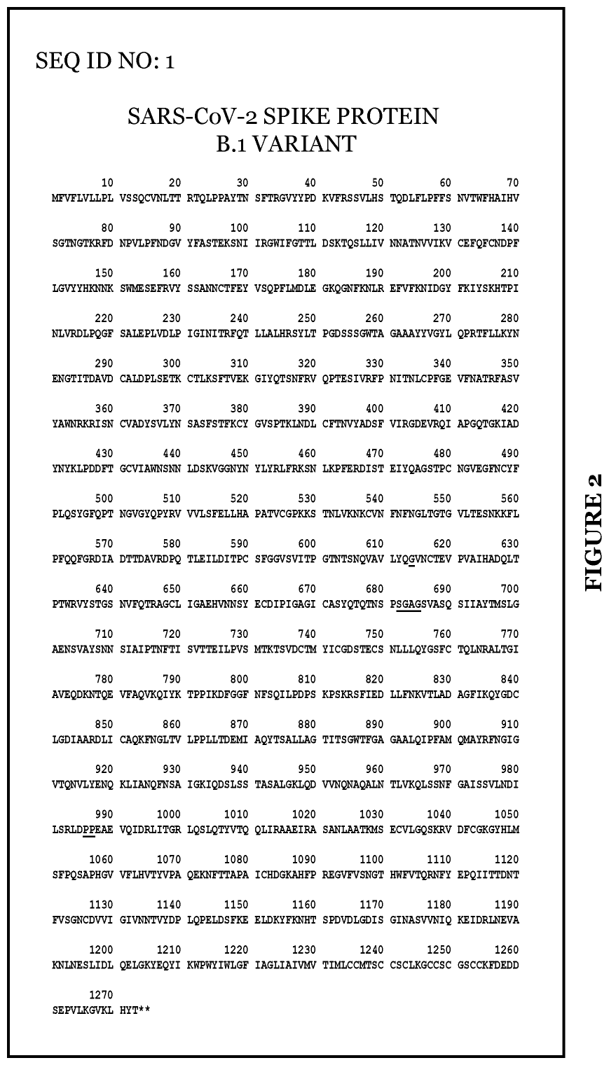

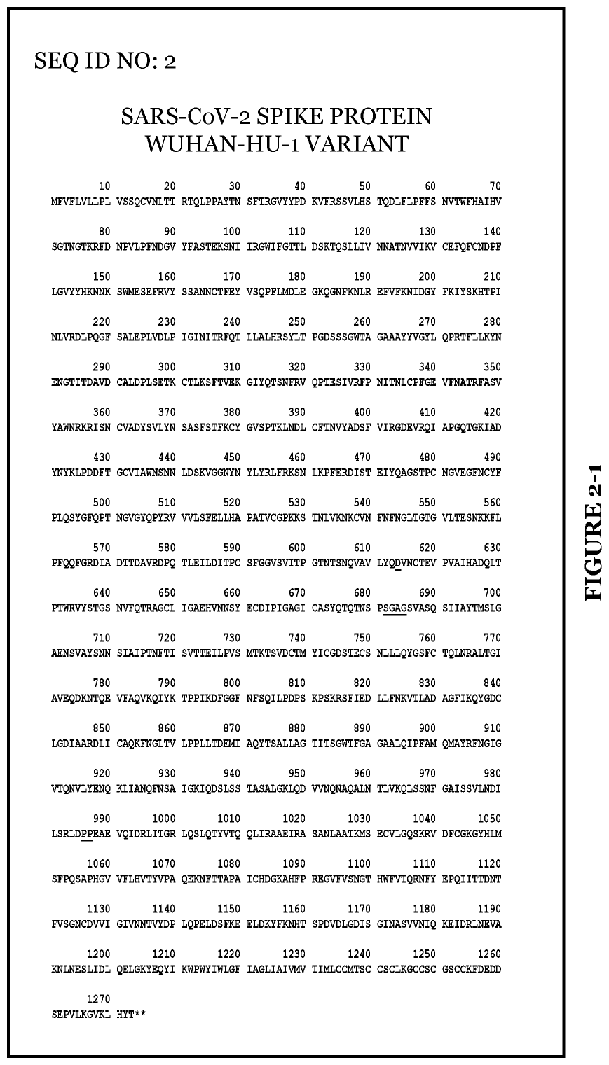

The plasmids capable of producing mRNA molecules that express the desired proteins like Corona virus spike protein antigens, Zoster virus protein antigens, Malaria protozoan antigens etc, when delivered to cells, were prepared by standard molecular biology techniques. These plasmids contained the Non Structural Proteins (NSP1 to NSP4) of the Venezuelan Equine Encephalitis Virus (VEEV) in cis with the coding sequences for the desired protein antigens or genes of interest. The NSP gene products help self-replication of the mRNA molecules inside the host cells. For example, when downstream to NSP sequences, SARS CoV-2 spike protein encoding sequence is simultaneously transcribed during self-replication and then translates the viral spike protein antigens in the host cell. Alongside replication and prolonged expression of spike protein generates the long lasting booster immune responses. All plasmids were routinely maintained in the E. coli cells using the standard protocols. A representative illustration is shown in the FIG. 1, that depicts the structural elements of the pVEE plasmids containing various genes of interest, like the D614G mutant of SARS-CoV-2 spike protein gene of B.1 variant as shown in FIG. 2, among other variants of the spike proteins or antigen relevant genes of interest from other viruses and organism. The various mutants or variants of the SARS-CoV-2 spike protein that may be used with respective the base spike protein sequence of Wuhan-Hu-1 variant [see FIG. 2-1] are shown TABLE 1. The pVEE vector is based of pMB1 plasmid known in the art. This vector contains T7 polymerase promoter element, 5′-UTR element, ORFs for the self-replicating non-structural proteins (NSP1-4), signal sequence, gene of interest like the spike protein, 3′ UTR element flowed by poly A tail element in a series, along with pMB1 base plasmid elements and kanamycin resistance marker.

TABLE 1

| VARIANTS | B.1 | B.1.351 | P.1 | B.1.1.7 | B.1.617 | B.1.618 | B.1.617.2 | AY.2 |
|---|---|---|---|---|---|---|---|---|
| AMINO ACID | D614G | D80A | L18F | 69Hdel | T95I | H49Y | T19R | T19R |
| CHANGES, | | D215G | T20N | 70Vdel | G142D | Y145del | G142D | V70F |
| INSERTIONS | | E484K | P26S | N501Y | E154K | H146del | E156G | G142D |
| AND/OR | | N501Y | D138Y | A570D | V382L | E484K | F157del | E156G |
| DELETIONS | | D614G | R190S | D614G | L452R | D614G | R158del | F157del |
| IN THE | | A701V | K417N | P681H | E484Q | | A222V | R158del |
| SPIKE | | | E484K | T716I | D614G | | L452R | A222V |
| PROTEIN | | | N501Y | S982A | E484Q | | T478K | K417N |
| OF THE | | | D614G | D1118H | P681R | | D614G | L452R |
| SARS-CoV-2 | | | H655Y | | Q1071H | | P681R | T478K |
| | | | T1027I | | E1072K | | D950N | D614G |
| | | | | | K1073R | | | P681R |
| | | | | | H1101D | | | D950N |
| | | | | | D1153Y | | | |

| VARIANTS | AY.1 | BA.1 | BA.2 | BA.2.12.1 | BA.4 | BA.5 |
|---|---|---|---|---|---|---|
| AMINO ACID | T19R | A67V | T19I | T19I | V3G | T19I |
| CHANGES, | 195I | H69del | L24del | L24del | T19I | L24del |
| INSERTIONS | G142D | V70del | P25del | P25del | L24del | P25del |
| AND/OR | E156G | T95I | P26del | P26del | P25del | P26del |
| DELETIONS | F157del | G142D | A27S | A27S | P26del | L452R |
| IN THE | R158del | V143del | G142D | G142D | A27S | A27S |
| SPIKE | W258L | Y144del | V213G | V213G | H69del | H69del |
| PROTEIN | K417N | Y145del | G339D | G339D | V70del | V70del |
| OF THE | L452R | N211del | S371F | S371F | G142D | G142D |
| SARS-CoV-2 | T478K | L212I | S373P | S373P | V213G | V213G |
| | D614G | Insertion 214EPE | S375F | S375F | G339D | G339D |
| | P681R | G339D | T376A | T376A | S371F | S371F |
| | D950N | S371L | K417N | D405N | S373P | S373P |
| | | S373P | N440K | R408S | S375F | S375F |
| | | S375F | D405N | K417N | T376A | T376A |
| | | K417N | R408S | N440K | D405N | D405N |
| | | N440K | S477N | L452Q | R408S | R408S |
| | | G446S | T478K | S477N | K417N | K417N |
| | | S477N | E484A | T478K | N440K | N440K |
| | | T478K | Q493R | E484A | L452R | S477N |
| | | E484A | Q498R | Q493R | S477N | T478K |
| | | Q493R | N501Y | Q498R | T478K | E484A |
| | | G496S | Y505H | N501Y | E484A | F486V |
| | | Q498R | D614G | Y505H | F486V | Q498R |
| | | N501Y | H655Y | D614G | Q498R | N501Y |
| | | Y505H | N679K | H655Y | N501Y | Y505H |
| | | T547K | P681H | N679K | Y505H | D614G |
| | | D614G | N764K | P681H | D614G | H655Y |
| | | H655Y | D796Y | S704L | H655Y | N679K |
| | | N679K | Q954H | N764K | N679K | P681H |
| | | P681H | N969K | D796Y | P681H | N764K |
| | | N764K | | Q954H | N764K | D796Y |
| | | D796Y | | N969K | Q954H | Q954H |
| | | N856K | | | N969K | N969K |
| | | Q954H | | | | A1020S |
| | | N969K | | | | |
| | | L981F | | | | |

### Example 2: Preparation of mRNA

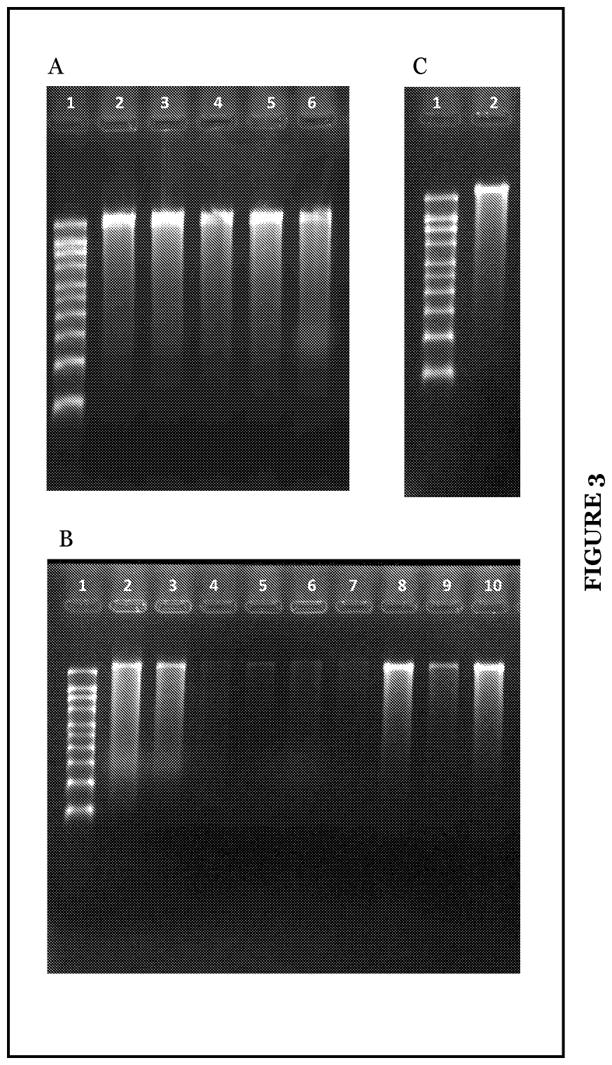

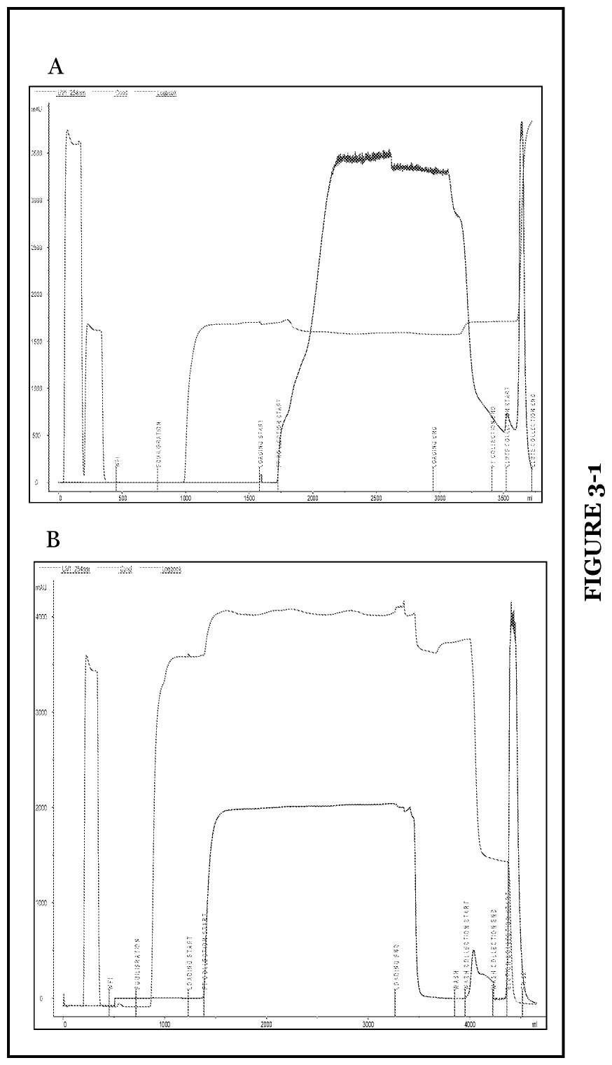

An in-vitro transcription [IVT] protocol was used for the preparation of mRNA molecules from a DNA template obtained from the plasmid described in Example 1. For a batch size of about 1200 mL to produce the mRNA of SARS-CoV-2 spike protein variant D614G [all the below Examples related to this mRNA molecule] the preparation was divided into three parts. In the first part, about 14 mL of highly pure water was taken in a 50-mL container, it was buffered with 1.5 mL of 1 M Tris-HCL, pH 8.0 solution. To this reaction mass about 0.975 mL of 1 M MgCl2 solution, about 10.5 mL of 25 mM of rNTPs each in solution and about 0.375 mL of 1 M dithiothreitol solution were added. Further to said reaction mass about 1.5 mL of 50 mM spermidine solution, about 7.5 mL of 250 ng/μL of template DNA solution, and about 0.75 mL of 1 ug/μL of inorganic pyrophosphatase solution were added. This was followed by about 0.21 mL of 1.5 μg/μL of RNAse inhibitor solution, and about 0.28 mL of 2 μg/μL of T7 polymerase solution. The resulting reaction mass of about 38 mL was gently mixed and incubated at about 32° C. on a shaker as about 100 RPM for about 4 hours. This part afforded robust synthesis of mRNA from the DNA template [see FIG. 3A Lanes 2 to 4, Lane 1 is RNA molecular weight ladder-first band is of 9 kb and the last is of 0.5 kb-which is same in all the experiments]. In the second part, at the end of reaction time, said 38-mL reaction mass was transferred to a 500-mL flask and to which about 350 mL highly pure water was added. This reaction mass was further supplemented with about 0.2 mL of 1 M CaCl2) solution and about 0.05 mL of 2.5 μg/μl DNAse solution. The resulting reaction mass of about 390 mL was gently mixed and incubated at about 32° C. on a shaker as about 100 RPM for about 30 minutes. This part completely removes the DNA template used in the first part [see FIG. 3A Lanes 5]. In the third part, at the end of reaction time, said 390-mL reaction mass was transferred to a 2000-mL flask and to which about 730 mL highly pure water was added. This reaction mass was further supplemented with about 58 mL of 1 M Tris-HCl, pH 8.0 buffer solution, about 0.225 mL of 1 M MgCl2 solution, about 2.5 mL of 1 M of KCl solution, about 12 mL of 100 mM of GTP solution and about 7.5 mL of 32 mM of S-adenosylmethionine solution and about 0.8 mL of 1 M of dithiothreitol solution. Further to it about 0.2 mL of 1.5 μg/μL RNAse inhibitor solution and about 0.75 mL of 2 μg/μL guanyltransferase solution were added. The resulting reaction mass of about 1200 mL was gently mixed and incubated at about 32° C. on a shaker as about 100 RPM for about 2 hours [see FIG. 3A Lanes 6]. This method afforded about 150 μg/mL of in-vitro transcribed mRNA within a period of about 7 hours. Herein, the mRNA synthesis was performed in sterile and nuclease-free conditions in clean-air condition. FIG. 3A shows the quality of mRNA produced as above in different parts.

### Example 3: Purification of mRNA

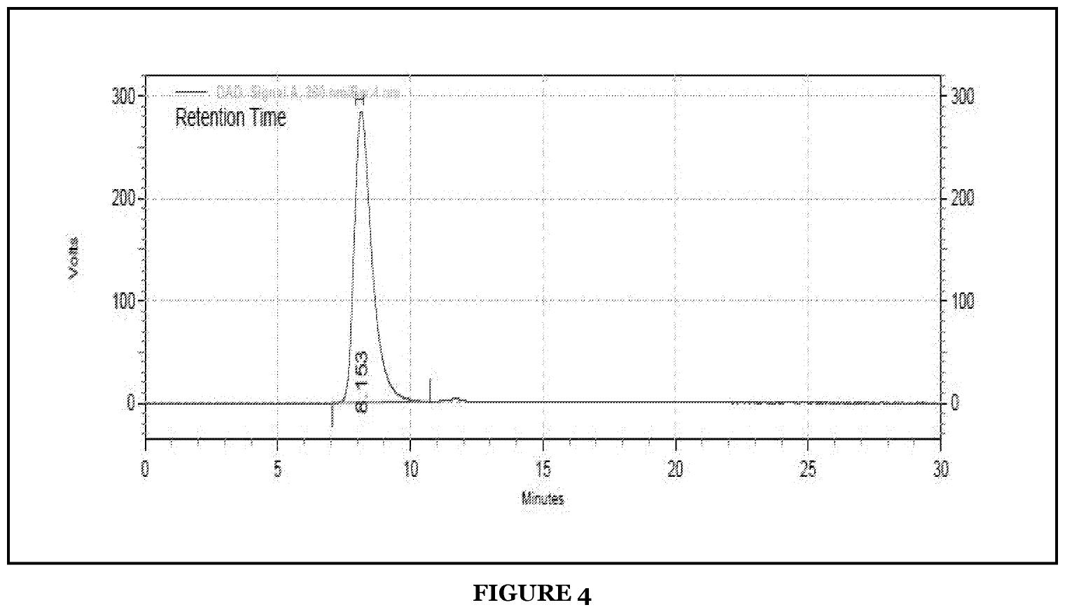

The reaction mass of Example 2 containing mRNA molecules was subjected to mRNA purification by chromatography and filtration methods. Herein, said of mass of about 1200 mL was supplemented with Tris-HCl and KCl stock solutions to achieve the final concentrations of 10 mM of Tris-HCl and 250 mM of KCl at pH 8.0. The first chromatographic step was used in flow through mode in which the impurities bind to the column while the mRNA molecules are collected in the flow through solution. Herein said diluted solution was subjected to a pre-equilibrated column having the octylamine based highly cross-linked agarose resin [CaptoCore 700-Cytiva] or similar resin matrix and the flow through fractions were collected, which contained the said mRNA molecules [see FIG. 3B, Lane 2 before first step, Lane 3 after first step; see FIG. 3-1 Chromatograph A]. The second chromatographic step was used in affinity mode in which the mRNA molecules bind to the column while the impurities come out with the flow through fractions. Herein, said mass of first step of about 1000 mL was supplemented with Tris-HCl, NaCl and EDTA stock solutions to achieve the final concentrations of 10 mM of Tris-HCl, 0.8 M of NaCl and 1 mM of EDTA at pH 8.0. Then subjected to a pre-equilibrated column having the oligo (dT) 25 based cross-linked poly(styrene-divinylbenzene) affinity resin [POROS—ThermoFisher] or similar affinity resin matrix and after completion of binding of mRNA molecules, was washed with a wash buffer containing 10 mM of sodium citrate and 0.8 M of NaCl at pH 6.5 and flow through fractions discarded [see FIG. 3B, Lane 4 to 7]. Next the bound mRNA molecules were eluted using an elution buffer containing 1 mM of sodium citrate at pH 6.5 [see FIG. 3B, Lane 8; see FIG. 3-1 Chromatograph B]. This step afforded about 800 mL eluate containing the purified mRNA molecules. The third step was used a diafiltration system with hollow fibre of 30 kDa molecular cut-off membrane to concentration the said eluate to a final retentate called RNA drug substance of about 500 mL containing about 200 μg/mL of said mRNA [see FIG. 3B, Lane 10]. Further said drug substance was filter sterilised using a 0.2 μmembrane filtration system and stored at −80° C. until used [see FIG. 3C, Lane 2]. Further the size exclusion HPLC analysis was performed on said drug substance using standard methods, said drug substance has retention time of about 8.15 minutes and showed more than 95% molecular purity [see FIG. 4]. The RNA drug substance was routinely diluted or concentrated to achieve mRNA concentration of between 0.1 to 1.5 mg/mL and stored at −80° C. until further used.

### Example 4: Preparation of Lipid Nano-Emulsion Particles/Nano-Carriers

The preparation of the nano-carriers or GNPs was achieved in a three-part process. In first part, the oil phase was prepared using all the hydrophobic substances that form the part of the said carrier. Herein, to prepare about 4 mL of said oil phase, about 3 g of DOTAP, about 3.7 g of sorbitan monostearate [SPAN-60] and about 3.75 g of squalene was mixed in a glass container. Said mixture was warmed ay about 65° C. till all the components got well mixed in homogenous consistency. In second part, about 3.7 g of polysorbate-80 was mixed with about 96 mL of 10 mM sodium citrate, pH 6.0 buffered solution, which was kept warm at 65° C. In third part, both the oil and aqueous phases were mixed under high shear mixer running at about 5000 RPM for about 15 minutes. Then this mixture was passed about 10 times through high pressure homogenizer at about 30,000 psi and primed with remaining aqueous phase affording about 100 mL of nano-carrier solution. The said nano-carrier solution optionally contained immunostimulating substances like MPL or GLA at an amount of about 0.5 μg/mL when desired. The nano-carrier GNP contained no MPL or GLA, while GNP-M contained MPL adjuvant and GNP-G contained GLA adjuvant [see TABLE 2].

### Example 5: Preparation Complexes of Nano-Carriers and mRNA Molecules

The adsorption of mRNA molecules onto said nano-carrier were performed very careful and precise process of mixing of said mRNA solution into said nano-carrier solution forming the stable complexes. Herein, the ratio of nitrogen [present on the DOTAP molecules] to phosphate [present on the RNA molecules; N:P ratio] was taken as a measure of association of said mRNA molecules to said nano-carrier particles as the mRNA molecules being negatively charged while the DOTAP molecules positively charged, it leading to adsorption of said mRNA molecules on said nano-carriers. To achieve the stable complexes of mRNA molecules with nano-carriers various N:P ratio between 1 and 150 DOTAP to RNA amounts were tried, keep the amount of RNA constant. This lead to the N:P ratios between 5 and 15 as ideal for obtaining the stable complexes of the mRNA adsorbed onto said nano-carriers. Therefore, to prepare said complexes, the said nano-carrier solution of Example 4 is diluted to about 6 mg/mL of DOTAP with 10 mM sodium citrate, pH 6.0 solution. Then, about 50 mL of this diluted nano-carrier solution was taken in a 1000-mL container and placed on an orbital shaker rotating at between 70 and 120 RPM. Then about 50 mL of mRNA solution [RNA drug substance] as prepared in Example 3 was added slowing using a syringe pump in about 5 minutes under the constant stirring condition at temperature of about 2-8° C. Then the mixture was allowed to form complexes at 2-8° C. for about 30 minutes, further diluted 1:1 with 20 mM sodium citrate, pH 6.0 containing about 540 mg/mL of sucrose and then the said complex solution was filtered through 0.45 μm and 0.22 μm membrane filters to obtain the sterile vaccine solution. To determine the amount of RNA molecules adsorbed onto the nano-carrier particles and changes in the properties of said nano-carrier, average particle sizes and particle size distribution parameters were measured by dynamic light scattering on Zetasizer Nano system [Malvern Panalytical]. TABLE 2 provides changes observed in said parameters of the nano-carriers upon adsorption of RNA molecules.

TABLE 2

| | | | | | GNP + | GNP-M + | GNP-G + |
|---|---|---|---|---|---|---|---|
| SN | PARAMETER | GNP | GNP-M | GNP-G | mRNA | mRNA | mRNA |
| 1 | Particle size [nm] | 51.00 | 40.71 | 50.50 | 63.34 | 56.98 | 72.28 |
| 2 | PDI | 0.271 | 0.196 | 0.184 | 0.224 | 0.207 | 0.180 |
| 3 | Zeta potential [mV] | 34.8 | 28.3 | 33.2 | 11.1 | 11.4 | 10.5 |
| 4 | RNA content [μg/mL] | — | — | — | 40.70 | 44.80 | 40.40 |
| 5 | Binding Efficiency [%] | — | — | — | 98.30 | 98.10 | 89.90 |

### Example 6: Determination of Integrity of RNA Adsorbed onto Nano-Carriers

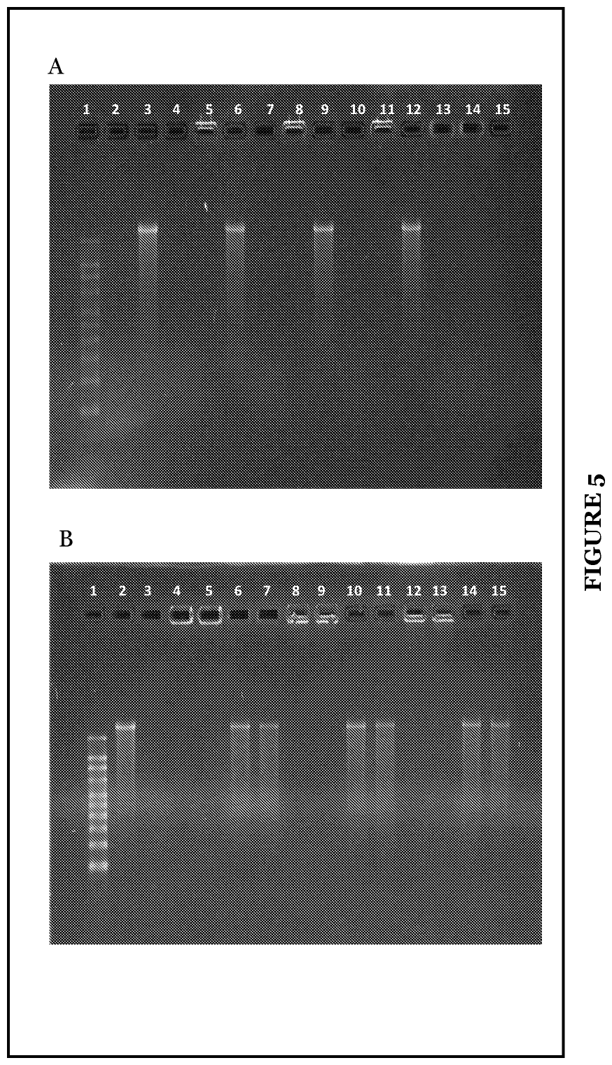

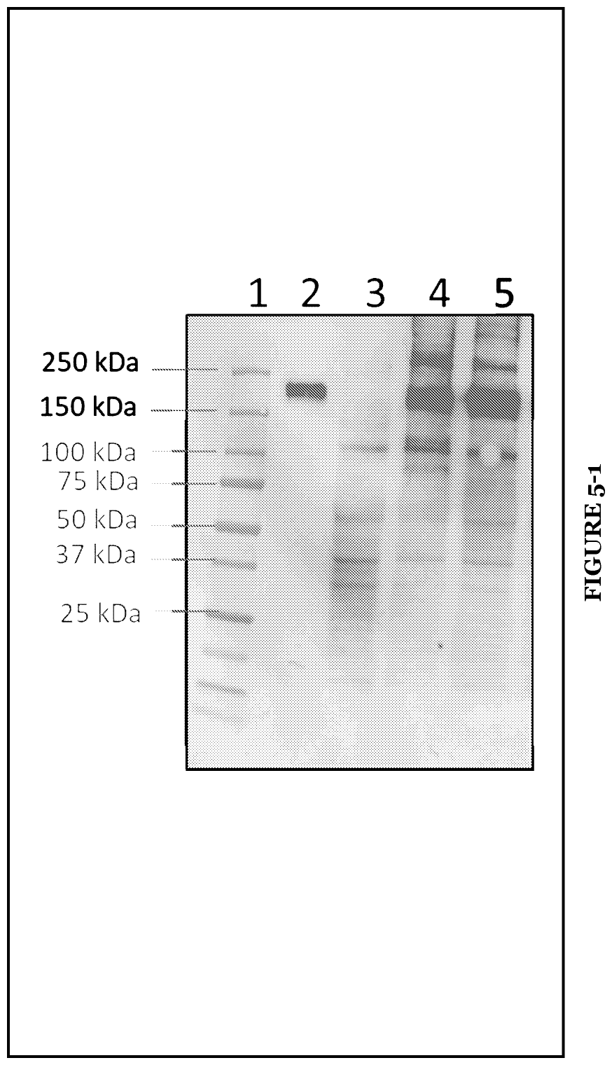

The integrity of the mRNA molecules as such or after extraction from the nano-carriers was determined by the formaldehyde denaturing agarose gel electrophoresis using methods known in the art. Briefly, mRNA samples were prepared in MOPS buffer with formaldehyde, ethidium bromide and a tracking dye like methylene blue by heating said mixture at about 70° C. for about 30 minutes. Then the samples were separated on 1% agarose gels upon completion of desired run of the samples in the gel and view under UV illumination and images preserved for the record. For the extraction of the mRNA molecules from the nano-carrier, said complexes were subjected to phenol-chloroform extraction. The results are shown FIG. 5A, wherein naked control mRNA runs on the gel corresponding to size of about 12 kb [Lane 3], while the RNA complexed with nano-carriers stay in the loading wells [Lanes 5-GNP, 8-GNP-M and 11 GNP-G], while the extracted mRNA molecules from nano-carriers also run at about 12 kb also [Lanes 6-GNP, 9-GNP-M and 12 GNP-G] without any degradation of its integrity, proving the integrity of the mRNA molecules in association with the nano-carriers.

The integrity of the mRNA was further tested with RNAse protection assays. Briefly, mRNA samples with or without nano-carriers were subjected to RNAse treatment and analysed by formaldehyde agarose gel electrophoresis. As shown in FIG. 5B, naked mRNA was treated with RNAse and showed complete degradation Lane 3 compared to untreated mRNA in Lane 2 control. In the case of mRNA complexed with GNPs, said nano-carriers protected the mRNA molecules, Lane 4 is GNP without treatment and Lane 5 is GNP treated with RNAse, in both the cases RNA remains attached to GNP particles and remains in the loading wells, showing protection of mRNA in complex with the nano-carrier. The mRNA extracted from the RNAse treated GNP particles [see Lanes 7, 11 and 15] and untreated GNP particles [see Lanes 6, 10 and 14] showed band size a bit above the 9 kb marker band indicating that said mRNA is intact without loss of integrity even after the RNAse treatment of the said complex.

### Example 7: RNA Quantification

The amount of mRNA was determined in different samples by the ultra-sensitive Qunati-IT RiboGreen RNA Assay Kit [ThermoFisher-Invitrogen] as per the manufacturers protocol. Briefly, when bound to free mRNA molecules the RiboGreen reagent has absorption and emission maxima at 500 nm and 525 nm, respectively. The detection sensitivity of this method is between 1 and 200 ng/ml of RNA in solution. Further the extracted RNA associated with lipid nano-carrier particles can also be easily detected using this method.

### Example 7A: Expression Protein in HEK Cells by RNA Adsorbed onto Nano-Carriers

To determine the potency of mRNA adsorbed or complexed onto the GNP carrier in-vitro protein expression analysis was performed in the HEK 293T cells. As shown in FIG. 5-1 western blot analysis recombinant Spike Protein was used a standard control [Lane 2], while untreated cells lysate was used as negative control [Lane 3]. The naked mRNA was transfected into the cells using Lipofectamine reagent [Lane 4], while mRNA adsorbed or complexed onto the GNP carrier added to the cell culture and incubated as disclosed [Lane 5]. The robust expression of the spike protein was observed mRNA transfected and complexed incubated samples was seen running as bands of about 170 kD. Herein HEK 293T cells were cultured for about 24 hours in 1 mL DMEM with FBS in cell culture plates and then treated as disclosed and incubated at 37° C. in presence of 5% CO2 for about 48 hours. After incubation, supernatant from the cell monolayer was discarded, and adhered cells were lysed with the 200 μL lysis buffer. The lysed solution was subjected to western blot using a SARS-CoV-2 (COVID-19) spike antibody for detection of the expressed spike protein. Sandwich ELISA with SARS-CoV-2 (COVID-19) spike antibodies was performed to estimate the expressed spike protein in the cell lysate.

### Example 8: Immunogenicity Studies

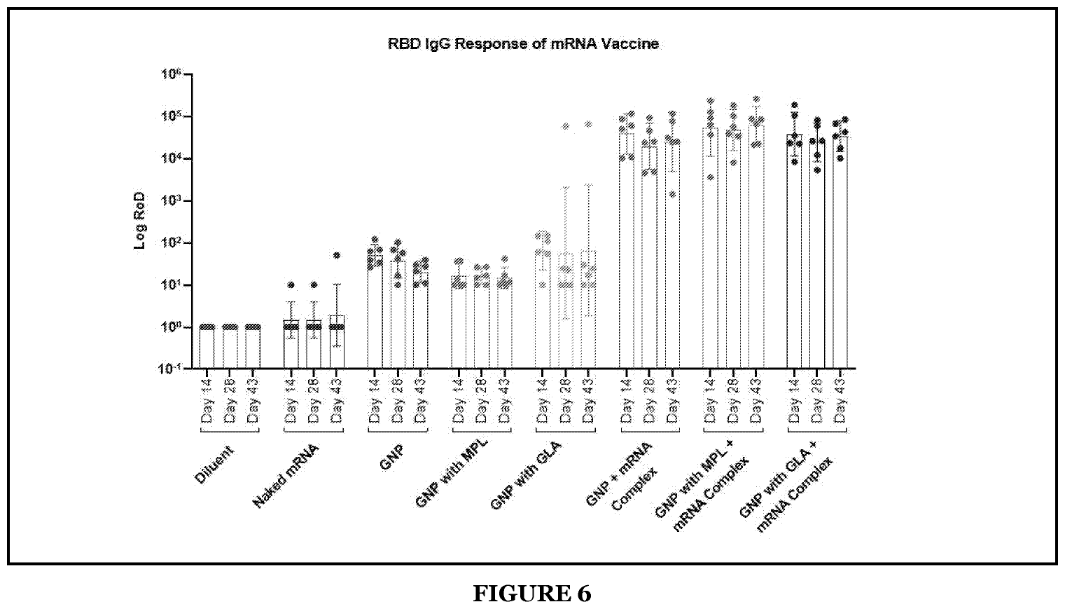

The vaccine solution obtained in Example 5 was subjected to the immunogenicity studies to determine the immunogen producing properties of the mRNA molecules adsorbed onto said nano-carriers. Herein, said vaccine solution or control solutions were injected to C57BL/6 or BALB/c mouse populations. About six mice were used per group as depicted in FIG. 6. The first control group was injected with the plain diluent or buffer solution only. The second control group was injected with naked mRNA equivalent to its amount present in the test vaccine solution, the nano-carrier control groups were injected with naked nano-carrier GNP or GNP-M or GNP-G equivalent to its amount present in the test vaccine solution. The first test group was injected with about 100 μL of test vaccine solution containing about 5 μg of mRNA complexed with GNP. The second test group was injected with about 100 μL of test vaccine solution containing about 5 μg of mRNA complexed with GNP-M. The third test group was injected with about 100 μL of test vaccine solution containing about 5 μg of mRNA complexed with GNP-G. All the studies groups were then tested for production of antibodies against the SARS-CoV-2 spike protein on the 14, 28 and 43 days after the injection of said materials. As shown in FIG. 6 the mouse group injected with the vaccine solutions [i.e., the mRNA complexed with GNP, GNP-M or GNP-G] showed marked increase in the production of antibodies against the spike protein compared to the naked mRNA injected group, while other control groups did not produce any antibody against the spike protein. The IgG antibodies in different groups were detected by standard protocols using ELISA assays. This data discloses the stability, immunogenicity and suitability for the clinical applications of said vaccine solution wherein mRNA is adsorbed onto the lipid nano-emulsion particles or nano-carriers.

### Example 9: Neutralising Antibody Studies

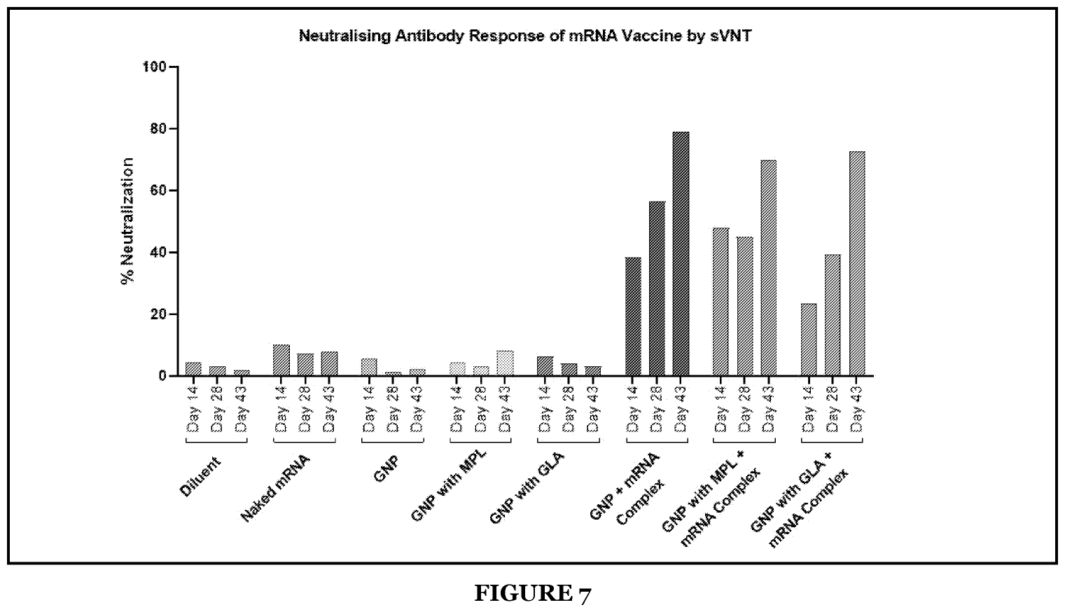

The SARS-CoV-2 surrogate virus neutralization test [sVNT] was performed using the cPass SARS-CoV-2 Neutralization Antibody Detection Kit [Genscript]. The assays were performed as per the manufacturer's protocol. Briefly, samples were diluted 10 times in dilution buffer. The diluted samples along with the positive and negative controls provided in the kit were incubated with equal volumes of 1000 fold HRP conjugated RBD supplied in the kit. Then the incubation was done at 37° C. for about 30 min. Then about 100 μL of all samples and controls are taken in the ACE-2 protein coated wells provided with the kit. The reactions were allowed in dark for about 15 min at 37° C. After 15 minutes the wells were washed 4 times before adding 100 μL of TMB substrate provided with the kit. The colours were allowed to develop for 15 min in dark before the reactions were stopped with 50 μL HCl solution provided with the kit. The plate was read at 450 nm in a plate reader. Percent inhibition is calculated as (1−(OD of sample/OD of negative control))×100%. The results are shown in FIG. 7. The mRNA when adsorbed onto different nano-carriers, like GNP, GNP-M or GNP-G showed marked increase in the production of SARS-CoV-2 spike protein neutralising antibodies.

1. A liquid formulation of RNA for administration in a human subject comprising:
a) an RNA capable of in-vivo expressing a protein, adsorbed onto;
b) a lipid nano-emulsion particles carrier; and
c) forming a stable RNA complex in which said RNA maintaining its integrity upon storage for an extended period of time.

2. The formulation of as claimed in claim 1, wherein concentration of said RNA, before formation of complex, is between 0.1 and 1.5 mg/mL.

3. The formulation of as claimed in claim 1, wherein said RNA is capable of expressing a variant of SARS-CoV-2 virus spike protein.

4. The formulation of as claimed in claim 1, wherein said RNA is prepared by an in-vitro translation process to a purity of between 70 and 95%.

5. The formulation of as claimed in claim 1, wherein the amount of said RNA is effective at producing said protein to a level that generates an effective immune response in said subject.

6. The formulation of as claimed in claim 1, wherein said amount of RNA is effective at reducing the risk of developing clinical signs of infection against which said RNA is targeted.

7. The formulation of as claimed in claim 1, wherein said RNA is a messenger RNA capable of self-replicating in HEK 293T cells.

8. The formulation of as claimed in claim 1, wherein said RNA has viral genetic elements for self-amplification in-vivo.

9. The formulation of as claimed in claim 1, wherein said nano-emulsion particles carrier comprises: a liquid lipid; a cationic lipid; a hydrophobic surfactant; a hydrophilic surfactant; and, optionally, an immunostimulatory adjuvant.

10. The formulation of as claimed in claim 1, wherein said carrier forms an oil in water emulsion.

11. The formulation of as claimed in claim 1, wherein said liquid lipid is squalene at a concentration between 3 and 40 mg/mL.

12. The formulation of as claimed in claim 1, wherein said cationic lipid is DOTAP at a concentration between 3 and 30 mg/mL.

13. The formulation of as claimed in claim 1, wherein said hydrophobic surfactant is sorbitan monostearate at a concentration between 3 and 40 mg/mL.

14. The formulation of as claimed in claim 1, wherein said hydrophilic surfactant is polysorbate 80 at a concentration between 3 and 40 mg/mL.

15. The formulation of as claimed in claim 1, wherein said immunostimulatory adjuvant is GLA or MPL adjuvant.

16. The formulation of as claimed in claim 1, wherein said carrier has a particle size between 50 and 70 nm.

17. The formulation of as claimed in claim 1, wherein said carrier has a zeta potential between 10 and 30 mV.

18. The formulation of as claimed in claim 1, wherein said carrier has particle dispersity index between 0.1 and 0.3.

19. The formulation of as claimed in claim 1, wherein said carrier is capable of adsorbing between 30 and 50 μg of mRNA per ug of said carrier.

20. The formulation of as claimed in claim 1, wherein said RNA complex has a particle size between 50 and 120 nm.

21-23. (canceled)
